(19) United States
(12) Patent Application Publication
(10) Pub. No.: US 2016/0069905 A1
(43) Pub. Date: Mar. 10, 2016

(54) Compositions and Methods for Detection of Defensins in a Patient Sample

(71) Applicant: The Research Institute at Nationwide Children's Hospital, Columbus, OH (US)

(72) Inventors: David Hains, Columbus, OH (US); Andrew Schwaderer, Blacklick, OH (US); John David Spencer, Dublin, OH (US)

(21) Appl. No.: 14/684,984

(22) Filed: Apr. 13, 2015

Related U.S. Application Data
(63) Continuation of application No. 14/347,737, filed on Mar. 27, 2014, now abandoned.
(60) Provisional application No. 61/541,318, filed on Sep. 30, 2011.

(51) Int. Cl. G01N 33/68

(52) U.S. Cl. CPC G01N 33/6893; G01N 2333/4721; G01N 2800/348

(57) ABSTRACT

Methods, devices and compositions for diagnosing, predicting and monitoring a urinary tract infection in a subject are described. The detection, prediction and monitoring of a urinary tract infection in a subject by detection of HD5 in the urine of a subject is described.

### CROSS REFERENCE TO RELATED APPLICATIONS

This application claims priority under 35 U.S.C. §119(e) to U.S. Provisional Application No. 61/541,318, filed Sep. 30, 2011, which is expressly incorporated by reference herein.

### FIELD OF THE INVENTION

The present invention relates to methods and compositions for diagnosing, predicting and monitoring a urinary tract infection in a subject. In particular, the present invention relates to the detection, prediction and monitoring of a urinary tract infection by detection of HD5 in the urine of a subject.

### BACKGROUND AND SUMMARY

The urinary tract, apart from the urethal meatus, is usually sterile despite its proximity with fecal flora. The precise mechanism by which the urinary tract maintains its sterility is not well understood. A subset of antimicrobial peptides (AMPs) have recently been shown to have an important role in maintaining urinary tract sterility. AMPs, a ubiquitous component of the innate immune system, are natural antibiotics produced by nearly all organisms. AMPs are small cationic proteins expressed by immune cells or epithelial cells either constitutively or via induction by invading pathogens. Certain AMPs exhibit a narrow antimicrobial spectrum, while others show broad-spectrum antimicrobial activity.

The defensins are the most well studied AMPs in humans. Collectively, defensins have broad-spectrum antimicrobial activity against gram-positive and gram-negative bacteria, viruses, fungi, and protozoa. Defensins are synthesized as preproproteins and undergo processing to become mature, biologically active peptides. Human defensins are classified into one of two families depending on their disulfide bridging pattern—the α-defensins or the β-defensins.

In the urinary tract, the β-defensins are widely expressed throughout the uroepithelium. Epithelial cells lining the kidney's loop of Henle, distal tubule, and collecting duct constitutively express human β-defensin 1 (hBD1). Although urinary levels of hBD1 are insufficient to kill invading bacteria, hBD 1 provides a fast-acting antimicrobial coating of tubular lumens and prevents infection by inhibiting bacterial attachment to the urothelium. Human β-defensin 2 (hBD2) is not constitutively expressed in healthy kidney tissue; however, hBD2 production is induced by infection. Unlike the β-defensins, the role of epithelial-derived α-defensins is not well described in the urinary tract.

Applicants describe herein methods and compositions to identify and quantitate the expression and production of defensins, e.g., HD5, in the urinary tract. HD5 is an epithelial-derived AMP that plays an important role in the innate immunity of the human uroepithelium preventing the translocation of invading pathogens into the circulation. The expression of the AMP human alpha defensin 5 (HD5) in the urinary tract in normal and infected subjects is described herein. The expression of DEFA5, the gene encoding HD5, in the urinary tract is also described. Applicants have shown, for the first time, the presence of HD5 in urine samples of infected subjects.

As described herein, the present kits and urine test strips provide advantages that are unavailable in prior compositions. The present kits and urine test strip's response to the presence of HD5 improves sensitivity and specificity, while at the same time reduce the instances of false positives resulting from contamination. In prior devices, the urine sample typically was required to be a catheter obtained or “clean catch” urine sample to ensure that there was no contamination. This problem is alleviated by the presently disclosed devices due to the response to the presence of HD5. The methods and compositions of the present invention provide the further advantage of allowing home testing by the subject.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE DRAWINGS

FIG. 1. shows the expression of DEFA5 in human kidney, ureter, and bladder. DEFA5 mRNA transcript levels were quantified by real-time PCR in non-infected kidney, ureter, bladder. Shown are the results of three independent samples. In the table below, the mean transcript levels are shown with the SEM. DEFA5 expression was significantly greater in the lower urinary tract (p=0.014).

FIG. 2 shows that HD5 expression increases with pyelonephritis. (A) DEFA5 mRNA transcript levels were quantified by real-time PCR in non-infected kidney tissue and in kidney tissue with pyelonephritis. Shown are the results for three independent samples. In the table below, the mean transcript levels are shown with the SEM. DEFA5 expression was significantly greater with pyelonephritis (p=0.019). (B) To confirm the increase in message is accompanied by an increase in HD5 protein production, cationic peptides from the same non-infected kidney tissues (NL) and kidney tissue with pyelonephritis (P) were subjected to SDS PAGE followed by Western immunoblot analysis. Each lane contained the equivalent of 800 μg of cationic protein. Silver stained PAGE gels (top panel) confirmed equal protein loading into each lane. Immunoblot analysis for GAPDH and HD5 (middle panel). Serial dilutions of proHD5 (200ng-70ng) were subjected to SDS PAGE followed by Western immunoblot analysis (bottom panel). (C) HD5 production was significantly greater with pyelonephritis (p<0.0001). In the table below, mean HD5 expression is shown (ng/mg tissue) with the SEM.

FIG. 3 shows that HD5 is expressed throughout the urothelium of the ureter and bladder. Immunohistochemistry demonstrates HD5 expression (brown/arrows) throughout the urothelium of the human bladder (A) and ureter (C). Immunostaining was most prominent in the luminal surfaces (brown/arrows). Immunostaining was not detected in the smooth muscle layers of the bladder or ureter (+). Negative control bladder (B) and ureter (D) showed no immunostaining. Magnification 20×.

FIG. 4 shows HD5 production in non-infected human kidney and kidney with pyelonephritis. Immunohistochemistry demonstrates HD5 production in isolated renal tubules (brown/arrows) in non-infected renal cortex (A) and medulla (C). With pyelonephritis, HD5 production increased in the renal tubules of the cortex (B) and medulla (D). The glomeruli (+) show no immunostaining in non-infected kidney samples and with pyelonephritis. Negative controls showed no immunostaining (not shown). Magnification 20×.

FIG. 5 shows tubular HD5 expression in states of sterility and infection. Human kidney labeled for HD5 (light area), nuclei ( ) and nephron specific markers ( ). Segment markers consisted of AQP-2 for collecting tubules (CT), THP for the loop of Henle (LOH), and AQP-1 for proximal tubules (PT). A/B: HD5 (light area) was produced in the collecting tubules (apical AQP-2 staining) of non-infected kidney tissue (A) and with pyelonephritis (B). Arrows indicate HD5 is produced in other nephron segments. C/D: HD5 (light area) was expressed in the loops of Henle (THP staining) in non-infected kidney tissue (C) and with pyelonephritis (D). E/F: HD5 (light area) shows minimal production in the proximal tubules (AQP-1 staining) of non-infected kidney tissue (E) and with pyelonephritis (F). Magnification 40×.

FIG. 6 shows that HD5 is present in infected urine samples. HD5 levels in sterile urine samples (n=15) and urine samples infected with uropathogenic E. coli (n=15). Cationic peptides from non-infected urine (NL) and infected urine (IN) were subjected to SDS PAGE followed by Western immunoblot analysis. Each lane contained the equivalent of 300 μg of urinary cationic protein. (A) Silver stained PAGE gels with 150 ng ProHD5 as standard to confirm equal protein loading into each lane. (B) Western blot analysis with 200 ng ProHD5 as standard.

FIG. 7 shows Urinary production of HD5 in infected urine samples. HD5 levels in sterile urine samples and urine samples infected with uropathogenic E. coli. The square boxes depict urinary proHD5 standardized to urine creatinine detected by ELISA assay using monoclonal antibody 8C8. The open circles depict urinary proHD5 and mature HD5 standardized to urine creatinine by immunoblot analysis. ProHD5 and mature HD5 were not detected in sterile urine samples using polyclonal HD5 antisera.

### DETAILED DESCRIPTION

In one embodiment, methods and compositions for diagnosing, predicting and monitoring a urinary tract infection are described. In one illustrative embodiment, methods of diagnosing, predicting, and monitoring a urinary tract infection by detection of defensins, e.g., a defensin protein and/or nucleic acid, such as HD5 (human alpha defensin 5) or DEFA5 (the gene encoding HD5), in a patient sample are disclosed. For example, methods of correlating the presence of certain defensins in urine with a urinary tract infection in a subject, e.g., cystitis, pyleonephritis, and urethritis, are described.

In one embodiment, a method is provided of determining if a patient is at risk for or has developed a urinary tract infection. The method comprises the steps of coupling at least an antibody to HD5, either by coupling the antibody to HD5 by direct binding to a support or via direct binding of the antibody to HD5 via a linker to the support, incubating the support coupled with the antibody to HD5 with a sample from a subject, and detecting HD5 bound to the antibody to HD5. In one embodiment, the method further comprises the step of diagnosing a urinary tract infection in the subject, or determining if the subject is at risk of developing a urinary tract infection, based on the amount of HD5 in the sample.

In another embodiment, a method of diagnosing a urinary tract infection is described. The method comprises the steps of providing a urine sample from a patient and reagents for detection of HD5 in the sample, wherein the reagents for detection are affixed to a solid support, contacting the urine sample with the reagents, detecting the presence or absence of HD5 in the urine sample using the reagents, and diagnosing a urinary tract infection in the subject, or determining if the subject is at risk of developing a urinary tract infection, based on the amount of HD5 in the sample.

In embodiments disclosed herein are methods, devices, and compositions for assessing if a subject is at risk of having or has a urinary tract infection, comprising: determining a HD5 level in a bodily sample of the subject; and comparing the HD5 level from the bodily sample of the subject to at least one predetermined value based on levels of HD5 in comparable bodily samples obtained from control subjects diagnosed as not having a urinary tract infection, wherein an increased level of HD5 in the bodily sample of the subject as compared to the at least one predetermined value based on levels of HD5 in the comparable bodily samples obtained from control subjects diagnosed as not having a urinary tract infection indicates that the subject is at risk of having or has a urinary tract infection. In some embodiments, the method further comprises the step of administering a therapeutic composition to a subject at risk of having or that has a urinary tract infection to reduce the risk of developing a urinary tract infection or to treat the urinary tract infection in the subject. In some embodiments, the bodily sample is urine.

In one embodiment, a kit for identifying a urinary tract infection in a subject comprising means for assaying a bodily sample of the subject for the detection of HD5 is described. In some embodiments, the bodily sample is urine.

In one embodiment, a urine test strip for identifying whether a subject is at risk of having or has a urinary tract infection is described, comprising: a reagent that provides a response to the presence of HD5 when immersed in, and removed from, a urine sample of the subject, wherein the response indicates whether the subject is at risk of having or has a urinary tract infection. In some embodiments, the response to the presence or absence of HD5 is a change of color. In some embodiments, the response to the presence of a determined threshold amount of HD5 is a change of color. The urine test strip may be produced via means known to those of ordinary skill in the art.

In one embodiment, methods and compositions for detecting the presence of HD5 in a patient urine sample are described. In some embodiments, full-length defensin proteins are detected. In some embodiments, a fragment or a portion of a defensin protein is detected. In one embodiment, methods of quantifying the amount of a defensin protein in the urine are described. The methods are not limited to a particular detection assay. Exemplary detection assays are described herein.

In some embodiments, HD5 is detected by binding of an antibody specific for the protein (i.e., an immunoassay). The present invention is not limited to a particular antibody. Any antibody (e.g., monoclonal or polyclonal) that detects defensins may be utilized.

Antibody binding is detected by techniques known in the art. For example, in some embodiments, antibody binding is detected using a suitable technique, including but not limited to, radio-immunoassay, ELISA, “sandwich” immunoassay, immunoradiometric assay, gel diffusion precipitation reaction, immunodiffusion assay, precipitation reaction, agglutination assay (e.g., gel agglutination assay, hemagglutination assay, etc.), complement fixation assay, immunofluorescence assay, protein A assay, and immunoelectrophoresis assay.

In some embodiments, a cytometric bead array assay is used (Quantum Plex kit, Bangs Laboratories; Cytometric Bead Array kit, BD Biosciences). These systems allow for multiple analyte detection with small volume samples. In other embodiments, a Luminex bead assay is used. In other embodiments, gold beads or dyed beads are used.

In illustrative embodiments, Enzyme-Linked Immunosorbant Assay (ELISA), affinity chromatography, or a Luminex®-based assay can be used to detect defensin proteins, such as the HD5 protein, in a patient body fluid sample. In one illustrative embodiment, the Luminex® assay utilizes microspheres (beads) that are dyed with fluorochromes and that are coupled to antigens to detect antibodies, in patient body fluids, to defensin proteins, such as the HD5 protein. In another embodiment, gold beads or dyed beads are utilized to detect HD5 protein in a patient body fluid. In another embodiment, the microspheres are coupled to antibodies to detect, in patient body fluids, defensin proteins, such as the HD5 protein. In this illustrative embodiment, the antibodies coupled to the microspheres can be polyclonal or monoclonal antibodies, but monoclonal anibodies are typically used. In some embodiments, the assay used to detect defensin proteins, is used to provide amounts of the defensin proteins.

The compositions and methods described herein contemplate the use of both monoclonal and polyclonal antibodies. Any suitable method may be used to generate the antibodies used in the methods and compositions of the present invention. The term “antibody” as used herein includes polyclonal or monoclonal antibodies or fragments that are sufficient to bind to an antigen. The antibody fragments can be, for example, monomeric Fab fragments, monomeric Fab′ fragments, or dimeric F(ab)′₂ fragments. Also within the scope of the term “antibody” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

The compositions and methods described herein detect the presence of certain defensins, e.g., HD5 or DEFA5, in a urine sample of a subject. Amounts of HD5 protein in the urine sample indicate the presence or absence of a urinary tract infection, including chronic infections, or risk of developing a urinary tract infection. A urinary tract infection can include an infection of the bladder, kidneys, and/or ureter, and the like, including cystitis, pyleonephritis, and urethritis. Illustrative urinary tract infections can include infections by one or more organisms, for example, organisms selected from Escherichia, Staphylococcus, Proteus Klebsiella, Enterococcus, Proteus, Morganella, Pseudomonas, or Candida, and the like.

It is to be understood that an amount of HD5, as used herein, generally refers to an amount of the mature HD5 peptide (AA63-94) or the combined amounts of one or more of the mature HD5 peptide, the HD5 propeptide (e.g. AA20-94), or other precursors to the mature peptide (e.g., partially processed forms AA36-94 and AA56-94, and the like).

In one embodiment, the step of detecting HD5 or DEFA5 in a sample includes detecting the presence or absence of any amount of HD5 or DEFA5, or can indicate detecting an amount of HD5 or DEFA5 in the sample over a threshold amount. In one embodiment, the presence of any amount of HD5 or DEFA5 in a patient sample indicates the presence of a urinary tract infection in the patient.

In one embodiment, an amount of HD5 in a urine sample of at least 50 ng HD5/mg urine creatinine indicates the presence of a urinary tract infection in the subject. Thus the threshold level of HD5 in a patient urine sample that indicates the presence of a urinary tract infection, or risk of developing a urinary tract infection, can be about 50 ng HD5/mg urine creatinine. In various illustrative embodiments, the threshold level of HD5 in a patient sample can be selected from the group consisting of at least about 10 ng HD5/mg urine creatinine, about 20 ng HD5/mg urine creatinine, about 30 ng HD5/mg urine creatinine, about 40 ng HD5/mg urine creatinine, about 50 ng HD5/mg urine creatinine, about 60 ng HD5/mg urine creatinine, about 70 ng HD5/mg urine creatinine about 80 ng HD5/mg urine creatinine, about 90 ng HD5/mg urine creatinine, and about 100 ng HD5/mg urine creatinine. In various illustrative embodiments, the threshold level of HD5 in a patient sample can range from about 25 to 75 ng HD5/mg urine creatinine, about 25 to 100 ng HD5/mg urine creatinine, and about 25 to 150 ng HD5/mg urine creatinine.

In one embodiment, the amount of HD5 in a sample that indicates the presence of a urinary tract infection (e.g., cystitis, pyelonephritis, and/or urethritis), or the risk of developing a urinary tract infection can be at least about 100 ng HD5/mg urine creatinine, about 125 ng HD5/mg urine creatinine, about 150 ng HD5/mg urine creatinine, about 200 ng HD5/mg urine creatinine, about 250 ng HD5/mg urine creatinine, about 300 ng HD5/mg urine creatinine, about 350 ng HD5/mg urine creatinine about 400 ng HD5/mg urine creatinine, about 450 ng HD5/mg urine creatinine, about 500 ng HD5/mg urine creatinine, about 750 ng HD5/mg urine creatinine, and about 1000 ng HD5/mg urine creatinine. In various illustrative embodiments, the amount of HD5 in a patient sample that indicates infection can range from about 50 to 250 ng HD5/mg urine creatinine, about 50 to 500 ng HD5/mg urine creatinine, about 100 to 500 ng HD5/mg urine creatinine, about 125 to 500 ng HD5/mg urine creatinine, about 100 to 750 ng HD5/mg urine creatinine, about 200 to 500 ng HD5/mg urine creatinine, about 200 to 750 ng HD5/mg urine creatinine, and about 100 to 1000 ng HD5/mg urine creatinine.

The compositions and methods disclosed herein can be used for both human clinical medicine and veterinary applications. Thus, the subject afflicted with the urinary tract infection, or at risk of developing a urinary tract infection, and in need of diagnosis or therapy can be a human, or in the case of veterinary applications, can be a laboratory, agricultural, or domestic animal. The methods and compositions described herein are suitable for use in both clinical and home testing settings. In one embodiment, testing is performed in a clinical (e.g., hospital or clinic) setting. In one embodiment, the subject can be male or female.

It is to be understood that samples of a bodily fluid can be obtained and/or tested using any means. For example, methods for collecting, handling and processing urine and other body fluids, are well known in the art and can be used in the practice of the present invention. In some embodiments, two or more consecutive or subsequent samples of a body fluid can be taken. Depending upon the circumstances, including the level of HD5 in a sample and the clinical condition of the subject, the subject's body fluid can be sampled daily, or weekly, or within a few weeks, or monthly or within a few months, semi-annually, annually, or within several years, and at any interval in between. Repeat sampling can be done at a period of time after treatment to detect any change in disease status. Sampling need not be continuous, but can be intermittent (e.g., sporadic). In some embodiments, it is not necessary to obtain and keep a sample of the bodily fluid from the subject. For example, in some embodiments, the subject can urinate onto a test strip.

Although generally the sample of a bodily fluid, such as urine, is obtained from a subject and tested by a laboratory or by a medical professional (for example using an automated urinalysis machine configured to test for HD5, or an HD5 testing kit, e.g. a urine dipstick based kit, or an ELISA based kit), home-testing kits are also contemplated. In one aspect, a kit for performing the methods of the invention, containing, for example, a device for detecting HD5 protein in the urine, and optionally including a positive control containing HD5 protein, and optionally including instructions, for example regarding the threshold levels of HD5 above which a diagnosis of a urinary tract infection can be made. The device in such kits can comprise, for example, an ELISA plate, a dipstick or a test strip to be dipped in a urine sample or to have a sample or urine applied thereto, or a stick on which the subject should urinate. In some embodiments, such devices are configured such that they give a positive result only if the level of HD5 exceeds a threshold level, such as one of the threshold levels described herein. Methods for making and using such devices are well known in the art. It is appreciated that such kits can be used by subjects themselves (e.g. home testing kits) or can be used by medical or laboratory staff.

The present compositions and methods are not limited to the detection of HD5 in urine. Any bodily fluid that contains elevated levels of HD5 correlated with the presence of a urinary tract infection, or risk of developing a urinary tract infection, may be utilized, including, but not limited to, blood, serum, lymph, saliva, sweat, mucous, any urinary tract secretion, washes, or lavage samples.

The present invention is further not limited to the direct detection of HD5 polypeptides or nucleic acids (e.g., mRNA). The present invention contemplates the detection of correlated polypeptides, nucleic acids, or compounds (e.g., DEFA5 mRNA, metabolites, etc.). In one embodiment, a test strip is provided as described herein, where the sample that can be analyzed is a patient body fluid, e.g., blood, serum, lymph, saliva, sweat, mucous, urine, any urinary tract secretion, washes, or lavage samples. In one embodiment, the test strip is a lateral flow test strip.

In one illustrative embodiment, test strips for determining the presence of a urinary tract infection comprise the following components: a) a sample pad—an absorbent pad onto which the test sample is applied; b) a conjugate or reagent pad—this comprises antibodies to HD5 conjugated to colored particles (colloidal gold particles, other colloidal metals, latex microspheres, dye sacs, fluorescent particles, magnetic particles, or the like); c) a reaction membrane—typically a hydrophobic nitrocellulose or cellulose acetate membrane onto which anti-HD5 antibodies are immobilized in a line across the membrane as a capture zone or test line (a control zone may also be present, containing antibodies specific for the conjugate antibodies); and d) a wick or waste reservoir—a further absorbent pad designed to draw the sample across the reaction membrane by capillary action and collect it. The components of the strip are usually fixed to an inert backing material and may be presented in a simple dipstick format or within a plastic casing with a sample port and reaction window showing the capture and control zones.

In one embodiment, for diagnosing urinary tract infections, kits are provided. The kits are useful for identifying, detecting, or quantitating defensins, e.g., HD5 or DEFA5, in a patient body fluid sample. In the embodiment where the kit is used to identify HD5 protein, the kit can contain a component with attached antibodies as described herein, e.g., a test strip, a container for collection of the bodily fluid, and the like. In one embodiment, the kit can contain components to detect defensin proteins in a patient sample, for example, antibody affinity matrices, ELISA plates, Luminex® beads, gold beads or dyed beads, polyclonal or monoclonal antibodies, color development reagents, buffers, and the like. In one embodiment, the reagents can remain in liquid form. In another embodiment, the reagents can be lyophilized. The kits can also contain instructions for use. In one embodiment, the kits contain all of the components necessary to perform a detection assay, including all controls, directions for performing assays, and any necessary software for analysis and presentation of results. In some embodiments, the kits contain an assay in a test strip format. In such embodiments, the detection reagent (e.g., antibody), as well as any control or secondary antibodies, are affixed to a solid support. In some embodiments, the solid support is a test strip suitable for dipping into a patient sample, e.g., a solution of urine

A calibration reagent (or multiple calibration reagents) can also be included in the test kit and “calibration reagent” means any standard or reference material containing a known amount of the defensin protein (i.e., HD5 protein). The patient sample and the calibration reagent (or multiple calibration reagents) are assayed under similar conditions. The protein concentration is then calculated by comparing the results obtained for the unknown patient sample with the results obtained for the calibration reagent(s).

Several illustrative embodiments of the invention are described by the following enumerated clauses:

1. A method for the detection of HD5, said method comprising the steps of:

(i) coupling at least an antibody to HD5, either by coupling the antibody to HD5 by direct binding to a support or via direct binding of the antibody to HD5 via a linker to the support;

ii) contacting the support coupled with the antibody to HD5 with a sample from a subject; and

iii) detecting HD5 bound to the antibody to HD5.

2. The method of clause 1, further comprising the step of diagnosing a urinary tract infection in the subject based on the amount of HD5 in the sample.

3. The method of clause 1 or 2, wherein the support is selected from the group consisting of a microtiter plate, a test strip, a slide, a wafer, a filter material, and a flow-through cell chamber consisting of polystyrene, polypropylene, polycarbonate, PMMA, cellulose acetate, nitrocellulose, glass, or silicon wafer.

4. The method of any one of the preceding clauses, wherein the support comprises a test strip.

5. The method of any one of the preceding clauses, wherein the amount of HD5 is determined by at least one of an immunoassay, a colorimetric assay, a turbidimetric assay, and flow cytometry.

6. The method of any one of the preceding clauses, wherein an amount of HD5 in the urine sample of at least 50 ng HD5/mg urine creatinine indicates the presence of a urinary tract infection in the subject.

7. The method of any one of the preceding clauses, wherein an amount of HD5 in the urine sample of at least 25 ng HD5/mg urine creatinine indicates the presence of a urinary tract infection in the subject.

8. The method of any one of the preceding clauses, wherein the urinary tract infection is cystitis.

9. The method of any one of the preceding clauses, wherein the urinary tract infection is pyelonephritis.

10. The method of any one of the preceding clauses, wherein the urinary tract infection is urethritis.

11. A device for the detection of HD5 in a sample of a subject, said device comprising a solid support coupled with an antibody to HD5.

12. The device of clause 11 wherein the solid support is selected from the group consisting of a microtiter plate, a test strip, a slide, a wafer, a filter material, and a flow-through cell chamber consisting of polystyrene, polypropylene, polycarbonate, PMMA, cellulose acetate, nitrocellulose, glass, or silicon wafer.

13. The device of clause 11 or 12 wherein the solid support is a test strip.

14. The device of clause 11, 12 or 13, wherein the sample is a urine sample.

15. The device of any one of clauses 12 to 14, wherein the test strip indicates the presence or absence of a urinary tract infection in the subject based on detecting an amount of HD5 in the sample.

16. The device of any one of clauses 12 to 15, wherein the amount of HD5 in the sample is determined by at least one of an immunoassay, a colorimetric assay, a turbidimetric assay.

17. The device any one of clauses 12 to 16, wherein the antibody is selected from the group consisting of a polyclonal antibody, a monoclonal antibody, and an antibody fragment.

18. The device of any one of clauses 12 to 17, wherein an amount of HD5 in the urine sample of at least 50 ng HD5/mg urine creatinine indicates the presence of a urinary tract infection in the subject.

19. The device of any one of clauses 12 to 18, wherein an amount of HD5 in the urine sample of at least 25 ng HD5/mg urine creatinine indicates the presence of a urinary tract infection in the subject.

20. The device of any one of clauses 12 to 19, wherein the urinary tract infection is cystitis.

21. The device of any one of clauses 12 to 20, wherein the urinary tract infection is pyelonephritis.

22. The device of any one of clauses 12 to 21, wherein the urinary tract infection is urethritis.

23. A kit comprising (i) components for the collection of a sample from a subject, (ii) components for detection of HD5 in the sample, and (iii) instructions for use.

24. The kit of clause 23, wherein the kit indicates the presence or absence of a urinary tract infection in the subject based on the amount of HD5 in the sample.

25. The kit of clause 23 or 24, wherein the amount of HD5 in the sample is determined by at least one of an immunoassay, a colorimetric assay, a turbidimetric assay, or flow cytometry.

26. The kit of any one of clauses 23 to 25 wherein the components for identification of HD5 in the sample include gold beads or dyed beads wherein the beads are coupled to antibodies to the HD5 protein.

27. The kit of any one of clauses 23 to 26, wherein the sample is a urine sample.

28. The kit of any one of clauses 23 to 27, wherein an amount of HD5 in the urine sample of at least 50 ng HD5/mg urine creatinine indicates the presence of a urinary tract infection in the subject.

29. The kit of any one of clauses 23 to 28, wherein an amount of HD5 in the urine sample of at least 25 ng HD5/mg urine creatinine indicates the presence of a urinary tract infection in the subject.

30. The kit of any one of clauses 23 to 29, wherein the urinary tract infection is cystitis.

31. The kit of any one of clauses 23 to 30, wherein the urinary tract infection is pyelonephritis.

32. The kit of any one of clauses 23 to 31, wherein the urinary tract infection is urethritis.

33. A method for assessing if a subject is at risk of having or has a urinary tract infection, the method comprising the step of:

a) determining if an HD5 level in a bodily sample of the subject is above a threshold value, wherein an HD5 level above the threshold value indicates the subject is at risk of having or has a urinary tract infection.

34. The method of clause 33, wherein the bodily sample is urine.

35. The method of clause 33 or 34 wherein the threshold value is at least 50 ng HD5/mg urine creatinine.

36. A kit for identifying a urinary tract infection in a subject comprising means for assaying a bodily sample of the subject for the presence of HD5.

37. The kit according to clause 36, wherein the bodily sample is urine.

38. A urine test strip for identifying whether a subject is at risk of having or has a urinary tract infection, comprising: a reagent that provides a response to the presence of HD5 when contacted with a urine sample of the subject, wherein the response indicates the subject is at risk of having or has a urinary tract infection.

39. The urine test strip according to clause 38, wherein the response to the presence or absence of HD5 is a change of color.

While certain embodiments of the present invention have been described and/or exemplified herein, it is contemplated that considerable variation and modification thereof are possible. Accordingly, the present invention is not limited to the particular embodiments described and/or exemplified herein.

### EXAMPLES

### Example 1

### Human Tissue and Urine Samples

Non-infected distal ureter and bladder tissue (n=4) was obtained from children undergoing ureteral re-implantation for reasons other than recurrent infection. Non-infected kidney samples (n=6) were obtained from patients undergoing nephrectomy for renal tumors. Tissue samples were free of microscopic signs of disease or inflammation. Kidney tissue from patients with chronic pyelonephritis was obtained from the Cooperative Human Tissue Network (n=6). Two independent pathologists confirmed the histopathologic diagnosis of pyelonephritis. Tissue samples were snap-frozen or preserved as neutral formalin-fixed paraffin-embedded sections. Sections of non-infected kidney tissue were dissected into cortex, medulla, or renal pelvis before storage (n=4).

Non-infected and infected urines samples were obtained from children presenting to the NCH emergency department or the nephrology clinic. The diagnosis of a UTI was made by a positive urine culture according to the American Academy of Pediatrics Guidelines. All infected urine samples had >10⁶ CFU/mL of E. coli. Urinary pH values ranged from 5.5 to 8.5 (mean urine pH 6.9). Urinary ionic composition was not measured. The urine samples were centrifuged to remove urine sediment, and protease inhibitor cocktail was added (Thermo Scientific, Rockford, Ill., USA).

### Example 2

### Ribonucleic Acid Isolation and Reverse Transcription

Total RNA was isolated from frozen tissue using the Promega Total RNA Isolation System (Promega, Madison, WI, USA). For cDNA synthesis, 4-8 μg of total RNA was reverse transcribed with Superscript III reverse transcriptase using an oligo-(dT) primer according to the supplier's protocol (Invitrogen, Carlsbad, Calif., USA).

### Example 3

### Cloning of Gene Specific Plasmids for Standard Curves

The cDNA encoding DEFAS was cloned into a 4-Topo plasmid vector (Invitrogen) according to manufacturer's instructions. Plasmids were sequenced to confirm that the correct constructs were obtained. Serial dilutions of gene specific plasmids were quantitated (by spectrophotometric absorbance at 260 nm and ethidium bromide staining agarose gel electrophoresis) and used in real-time PCR to generate standard curves for each reaction.

### Example 4

### Quantitative Real-Time PCR

Quantitative real-time PCR was performed using single-stranded cDNA from human kidney, ureter, and bladder tissue with specific oligonucleotide primer pairs using the 7500 Real-Time PCR System (Applied Biosystems, Carlsbad, Calif., USA). PCR exon junction spanning primers were designed and sequences were confirmed using DNAstar® Laser Gene SeqBuilder (DEFA5 forward primer: 5′- TCC CTC CTG CAG GTG ACC CCA-3′ and DEFA5 reverse primer 5′-GTG GCT CTT GCC TGA GAA CCT GA-3′).

Briefly, cDNA corresponding to lOng RNA served as a template in a 25 μl reaction containing 2.0 μM of each primer and lx Light-Cycler-Fast Start DNA Master SYBR Green mix. The PCR conditions were: initial denaturation at 95° C. for 10 minutes, followed by 40 cycles with each cycle consisting of denaturation at 94° C. for 30 seconds, annealing at 64° C. for 30 seconds, and extension at 72° C. for 30 seconds. The cycle-to-cycle fluorescence emission was monitored at 530 nm and analyzed using 7500 Software V2.0.3 (Applied Biosystems). Gene specific plasmid standards were included with every set of reactions. As a positive control, terminal ileum RNA was included with each set of reactions, and results were compared to previously published standards.

To confirm PCR amplification of the intended product, a representative sample was analyzed by electrophoresis on a 1.5% agarose gel. The products were visualized by ethidium bromide staining and compared to DNA size standards to confirm anticipated product size. In addition, a melting temperature profile curve of every PCR reaction was determined at the end of each reaction.

### Example 5

### HD5 Antibodies

The HD5 propeptide (AA20-94) and partially processed forms (AA36-94 and 56-94) were identified using a commercially available mouse monoclonal (8C8) anti-HD5 antibody (Abcam). The HD5 propeptide and the mature peptide (AA63-94) were identified using previously described rabbit polyclonal antiserum.

### Example 6

### Immunohistochemistry

Following deparaffinization, rehydration, and antigen retrieval, a biotin block and a serum-free protein block were performed (Superblock, ScyTek Laboratories, Logan, Utah, USA). The slides were incubated overnight at 4° C. with monoclonal mouse HD5 (8C8) antibody (1:200) (Abcam) or rabbit polyclonal antiserum (1:500) followed by anti-polyvalent biotinylated antibody and UltraTek Streptavidin/HRP (ScyTek Laboratories). Sections were developed using 0.1% diaminobenzidine tetrachloride with 0.02% hydrogen peroxide and counterstained with hematoxylin. Negative controls sections were incubated with non-immune serum in place of HD5 antibody.

### Example 7

### Immunofluorescence

Double-labeled immunofluorescence was performed to help localize HD5 expression in the kidney. The collecting duct was double-labeled for principal cells with goat polyclonal anti-human aquaporin-2 antibody (Santa Cruz Biotechnology, Santa Cruz, Calif., USA). The loop of Henle was double-labeled with mouse polyclonal anti-human uromodulin antibody (Sigma-Aldrich) and the proximal tubule was double-labeled with goat polyclonal antihuman aquaporin-1 (Santa Cruz). Rhodamine donkey polyclonal anti-goat (Jackson ImmunoResearch Laboratories, West Grove, Pa., USA), rhodamine goat anti-mouse (Jackson ImmunoResearch Laboratories), and FITC donkey polyclonal anti-rabbit (Santa Cruz) served as the secondary antibodies.

All sections were prepared as outlined above. They were incubated with a mixture of mouse antisera against HD5 (1:200) (Abcam), AQP-2 (1:400), uromodulin (1:500), AQP-1 (1:400) at room temperature for 90 minutes. The secondary antibody was applied for 90 minutes at room temperature and the sections were mounted using mounting media with DAPI. Non-immune serum was used as a negative control. The slides were examined with a Leica DM4000B microscope and digitally photographed using Spot RT camera/software (Diagnostic Instruments, Sterling Heights, Mich., USA).

### Example 8

### Immunoblot Analysis

Sections of the same kidney specimens used for quantitative real time PCR analysis (3-6 mg wet weight) were pulverized using a mortar and pestle in liquid nitrogen and dissolved in RIPA buffer with protease inhibitors. Urinary proteins were extracted from human kidney and urine samples using the PROTEOSPIN™ Urine Protein Concentration Micro Kit (Norgen Biotek Corporation, Thorold, ON, Canada). Kidney and urine protein concentrations were quantified using a modified Bradford Assay and confirmed using an OD₂₈₀ₙₘ reading. Kidney specimens (3-6 mg wet weight) were pulverized using a mortar and pestle in liquid nitrogen and dissolved in RIPA buffer (Sigma-Aldrich) with protease inhibitors. Equal concentrations of urinary protein or kidney tissue were loaded onto 18% sodium dodecyl sulfate gradient gels and subjected to electrophoresis. To ensure equal protein loading, a silver stain was performed.

After electrophoretic separation, proteins were transferred onto a nitrocellulose membrane. The membranes were blocked in 5% fat-free milk for 30 to 60 minutes and incubated in a 1:1,000 dilution of rabbit polyclonal HD5 antiserum overnight. After washing, the secondary antibody, an anti-rabbit horseradish peroxidase-conjugated anti-rabbit IgG diluted 1:10,000 (Cell Signaling Technology, Danvers, Mass., USA), was applied for 1 hour at room temperature. Immunoblots from kidney tissues were also probed with anti-GAPDH antibody (Sigma Aldrich) for two hours at room temperature and then incubated with the secondary antibody described above. The proteins were visualized using and ECL detection system and chemiluminescence film according to the manufacturer' s instructions (BioExpress, Kaysville, Utah, USA). HD5 was quantitated by comparing resulting band intensities with a serial dilution of recombinant standard proHD5 protein (Peptides International, Louisville, Ky., USA).

HD5 was quantitated by comparing resulting band intensities with a serial dilution of recombinant standard proHD5 protein (Peptides International, Louisville, Ky., USA). Kidney HD5 concentrations were standardized to wet tissue weight. HD5 concentrations were divided by urine creatinine to establish standardized urine HD5-to-creatinine ratios (μg/mg) to account for urine dilution. Urine creatinine concentrations were determined using the Oxford Biomedical Research creatinine microplate assay (Rochester Hills, Mich., USA).

### Example 9

### ELISA

96-well flat-bottomed plates (Maxisorb, Nunc™, Rochester, N.Y., USA) were coated overnight at 4° C. with mouse monoclonal antibody to HD5 (3 μg/mL) (Abcam). After blocking with synthetic blocking buffer (Kem-En-Tec Diagnostics, Denmark), 100 μL standards and/or urine samples were added to the wells and incubated for 2 hours at room temperature. Serial dilutions of recombinant HD5 protein served as the standards (Novus Biologicals). Following incubation with a biotinylated (Lightning-Link Biotin Antibody Labeling Kit, Novus Biologicals) mouse monoclonal antibody for 2 hours at room temperature, streptavidin-horse radish peroxidase (Biolegend, San Diego Calif., USA) was added for 30 minutes. After incubation with TMB substrate solution for 15 minutes (Kem-En-Tec Diagnostics), the reaction was terminated with STOP solution (Cell Signaling Technology, Danvers, Mass., USA) and read at a wavelength of 450 nm. HD5 concentrations from the ELISA assay were divided by urine creatinine to establish standardized urine HD5-to-creatinine ratios (μg/mg) to account for urine dilution as described above.

### Example 10

### DEFA5 MRNA is Constitutively Expressed in Human Bladder, Ureter, and Kidney and Expression Increases with Pyelonephritis

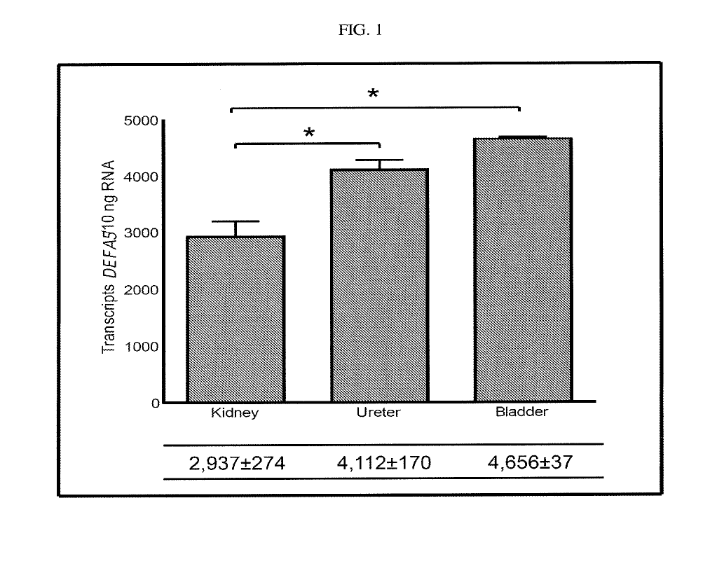

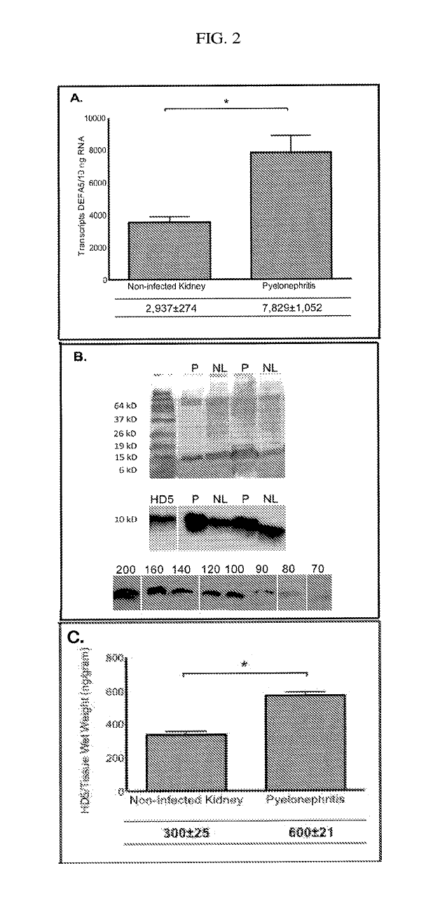

Quantitative real-time PCR demonstrates that all tested bladder, ureter, and kidney specimens without infection constitutively expressed DEFA5. DEFA5 expression was significantly greater in the lower urinary tract than the upper urinary tract (p=0.014). In the bladder (n=4), mean DEFA5 expression was 4,656±37 transcripts per lOng RNA and in the ureter (n=4) mean DEFA5 expression was 4,112±170 transcripts per lOng RNA (FIG. 1). In the kidney (n=6), mean DEFA5 expression was 2,937±274 transcripts per lOng RNA. DEFA5 expression was analyzed separately in the renal cortex, medulla, and pelvis. DEFA5 expression did not significantly vary by kidney region (p=0.45). In patients with pyelonephritis (n=6), mean DEFA5 expression in the kidney increased to 7,829±1,052 transcripts per lOng RNA (p=0.019) (FIG. 2A).

The quantitative real-time PCR results demonstrate that DEFA5 is constitutively expressed in the human kidney, ureter, and bladder. Notably, DEFA5 expression increases from the upper urinary tract to the lower urinary tract—following the flow of the urinary stream and the increasing closeness to the microbiota. In addition, the real-time PCR data demonstrate that DEFA5 expression in the kidney is significantly up-regulated with infection.

### Example 11

### DEFA5 Expression and HD5 Peptide Expression Increase with Pyelonephritis

Quantitative real-time PCR analysis performed on kidney tissues with pyelonephritis demonstrated a significant increase in DEFA5 expression compared to non-infected kidney tissues. With pyelonephritis (n=6), mean DEFA5 expression increased to 7,829±1,052 transcripts per lOng RNA (p=0.019) (FIG. 2A).

To further evaluate this increase in expression with pyelonephritis, we performed immunoblot analysis on the same kidney tissue used for real-time PCR analysis to evaluate for concurrent increases in HD5 peptide production (FIG. 2B, middle panel). Immunoblot analysis, using polyclonal HD5 antisera, demonstrated significantly greater HD5 peptide production in kidney tissues with pyelonephritis (n=6) compared to non-infected kidney tissues (n=6). Non-infected kidney tissues expressed 300±25 ng HD5/gram wet tissue weight while kidney tissue with pyelonephritis expressed 600±21 ng HD5/gram wet tissue weight (p<0.0001) (FIG. 2C). Blots were re-probed with GAPDH to serve as a loading control (FIG. 2B, middle panel) and a silver stain was also performed to confirm equal protein loading (FIG. 2B, top panel).

### Example 12

### HD5 Peptide is Expressed Throughout the Human Kidney and Urinary Tract

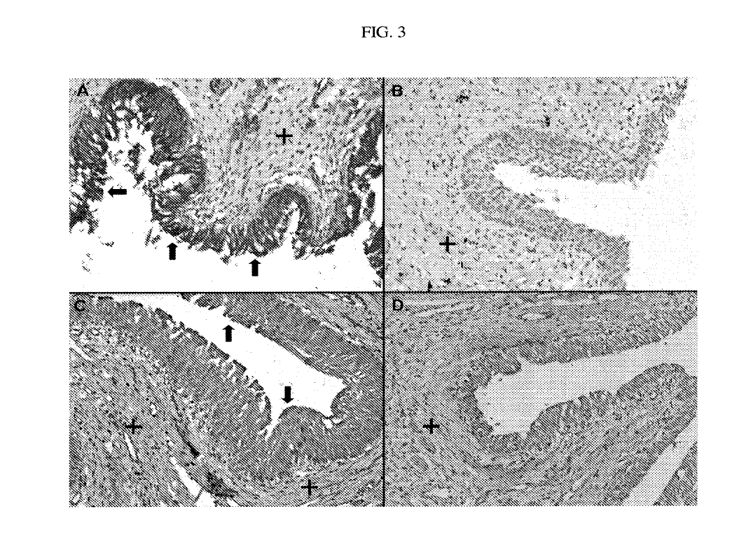

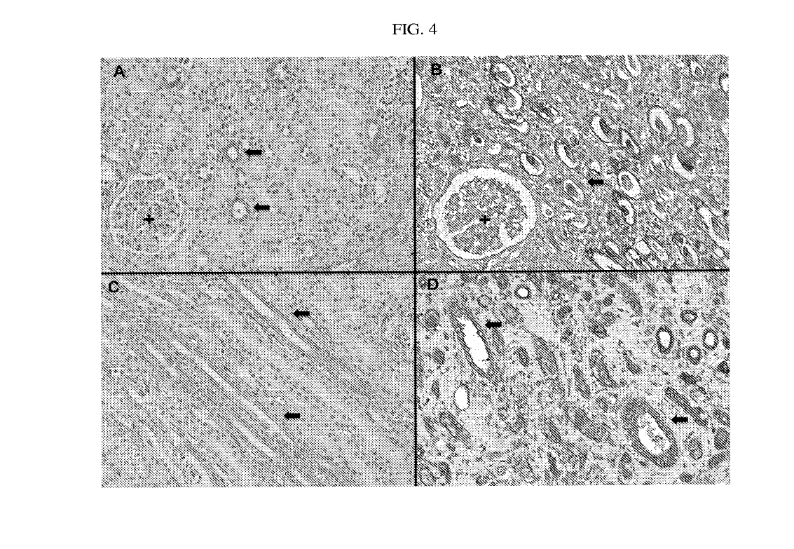

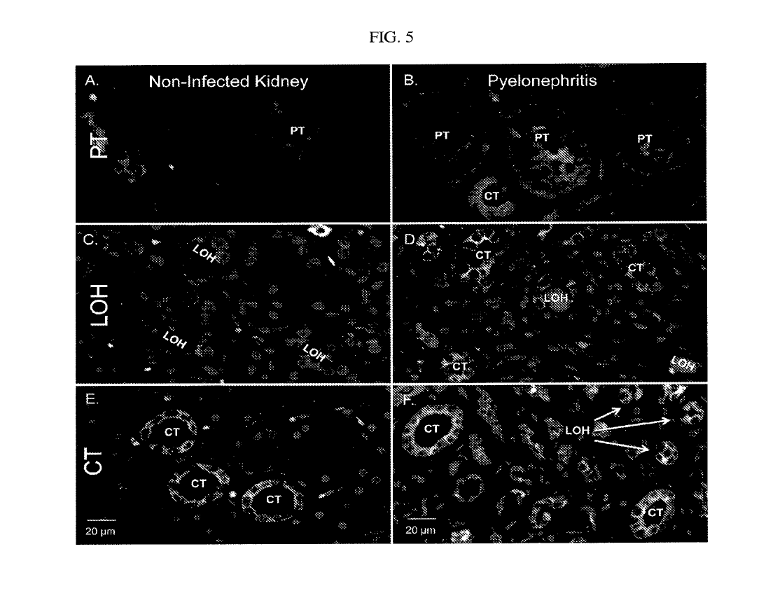

Immunostaining was performed to localize HD5 production in the urinary tract. Immunohistochemistry (IHC) showed that HD5 immunoreactivity was present throughout the urothelium of the ureter and bladder of all investigated specimens (n=4) (FIG. 3). IHC also showed that HD5 was produced in the renal cortex and medulla of all specimens (n=6) (FIG. 4). Immunofluorescence (IF) demonstrated that HD5 expression was greatest in the distal nephron and the collecting tubules (FIG. 5). The interstitium and glomeruli did not show HD5 immunoreactivity. The immunostaining shown in FIGS. 3-5 was performed using a mouse monoclonal anti-HD5 antibody (8C8) that recognizes the HD5 propeptide (Abcam). Results were similar when using rabbit polyclonal anti-HD5 antibody that recognizes the HD5 propeptide and mature peptide (data not shown) suggesting that HD5 is primarily stored as propeptide. IHC demonstrated that proHD5 is a major form of HD5 in the kidney and because both mature and proHD5 are detected in the urine.

### Example 13

### HD5 Peptide Expression Increases Throughout the Nephron with Pyelonephritis

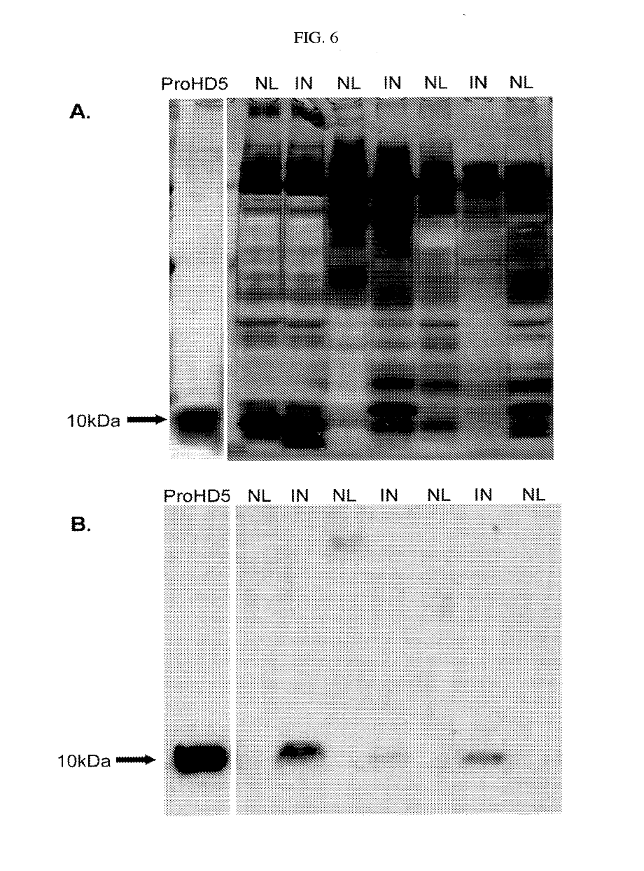

Results indicate a marked increase in HD5 immunostaining in specimens with pyelonephritis. HD5 immunoreactivity increased throughout the proximal nephron, the distal nephron, and the collecting tubules (FIG. 5). As in non-infected specimens, the glomeruli and interstitium showed no HD5 expression with infection. Negative controls showed no HD5 immunoreactivity. Immunoblot analysis, using polyclonal HD5 antisera, demonstrated significantly greater HD5 peptide production in kidney tissues with pyelonephritis (n=6) (FIG. 6A and B) compared to non-infected kidney tissues (n=6) (p<0.0001).

Immunostaining shows that HD5 is uniformly produced throughout the urothelium of the ureter and bladder. Since, the vast majority of UTIs result from fecal flora that ascend into the bladder, these results show that HD5 expression is present in locations where microbial exposure occurs most frequently, thus being positioned to prevent an ascending microbial infection. In the kidney, HD5 is primarily produced in the distal nephron and collecting tubule. These findings show that HD5 is produced in locations where it will be positioned to have optimal antimicrobial activity. The immunostaining and immunoblot analysis complement the real-time PCR data by demonstrating that HD5 peptide production increases with pyelonephritis.

### Example 14

### HD5 Peptide is Secreted into Human Urine in Measurable Concentrations

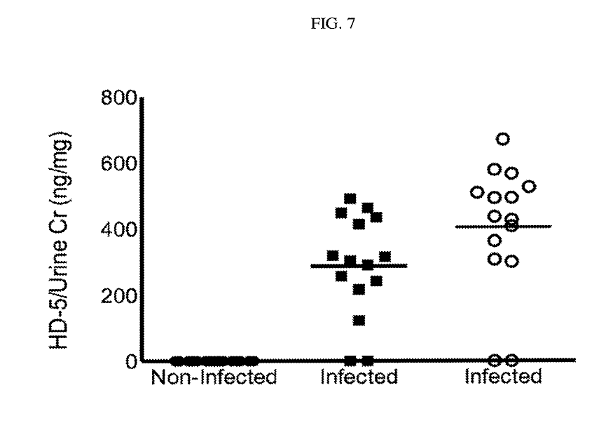

Immunoblot analysis, using rabbit polyclonal antiserum that detects the precursor proHD5 and further processed forms, identified measurable levels of HD5 in 13 of the 15 tested urine samples infected with uropathogenic E. coli (FIG. 7). When present, HD5 levels, normalized to urine creatinine, ranged from 299.8-669.7±30 ng HD5/mg urine Cr (110.67 ng/mL—276.67 ng/mL), which corresponds to 11.10-27.67 nmol/L. In non-infected urine samples (n=15), HD5 was inconsistently detected around detection limit (<50 ng). Enzyme linked immunosorbant assay (ELISA) on the same urine samples, using the mouse monoclonal antibody (8C8) that detects only the precursor proHD5, detected measurable levels of proHD5 in 13 of the 15 infected samples tested. Urinary proHD5 concentrations ranged from 122.78-490.0 ±0.03 ng HD5/mg urine Cr (30.02 ng/mL—200.5 ng/mL) when present (FIG. 7).

Immunoblot and ELISA analysis also demonstrate that HD5 is secreted into the urine at low levels. Given the size (8.1 kDa and 3.7 kDa) and positive charge of proHD5 and fully processed HD5, it is possible that some urinary HD5 peptide originates, at least in part, from plasma filtrate. Yet, there is little evidence suggesting that HD5 persists in the plasma. Additionally, to appear in the urine, HD5 would need to escape the efficient peptide absorption mechanisms in the proximal tubule. Finally, the urine samples used underwent centrifugation before the assays were performed, removing cellular sources of HD5.

These data demonstrate the presence of HD5 in infected urine, while nearly absent in non-infected urine. The amount of HD5 protein-to-creatinine ratio in a urine sample is a rapid, sensitive, and inexpensive biomarker for a urinary tract infection (e.g. pyelonephritis, and the like). This test can be used to further inform clinicians on stratifying urinary tract infections for work-up and management.

1-26. (canceled)

27. A method for determining if a subject has a urinary tract infection, said method comprising the steps of:
i) coupling at least an antibody to human alpha defensin 5 (HD5) to a support, by coupling the antibody to HD5 to the support by direct binding to the support or via binding of the antibody to HD5 via a linker to the support;
ii) contacting the support coupled with the antibody to HD5 with a urine sample from the subject;
iii) determining the amount of HD5 in the sample by detecting HD5 bound to the antibody to HD5; and
iv) diagnosing a urinary tract infection in the subject based on a threshold amount of HD5 in the sample.

28. The method of claim 27, wherein the support is selected from the group consisting of a microtiter plate, a test strip, a slide, a wafer, a filter material, and a flow-through cell chamber consisting of polystyrene, polypropylene, polycarbonate, poly(methyl methacrylate) (PMMA), cellulose acetate, nitrocellulose, glass, or silicon wafer.

29. The method of claim 27, wherein the support comprises a test strip.

30. The method of claim 27, wherein the amount of HD5 is determined by at least one of an immunoassay, a colorimetric assay, a turbidimetric assay, and flow cytometry.

31. The method of claim 27, wherein the threshold amount of HD5 in the urine sample is at least 50 ng HD5/mg urine creatinine.

32. The method of claim 27, wherein the urinary tract infection is cystitis or pyelonephritis, or urethritis.

33. A device for determining the presence of a urinary tract infection in a subject by the detection of an amount of HD5 in a urine sample from the subject, said device comprising a solid support coupled with an antibody to HD5.

34. The device of claim 33 wherein the solid support is selected from the group consisting of a microtiter plate, a test strip, a slide, a wafer, a filter material, and a flow-through cell chamber consisting of polystyrene, polypropylene, polycarbonate, poly(methyl methacrylate) (PMMA), cellulose acetate, nitrocellulose, glass, or silicon wafer.

35. The device of claim 33 wherein the solid support is a test strip.

36. The device of claim 35, wherein the amount of HD5 in the sample is determined by at least one of an immunoassay, a colorimetric assay, or a turbidimetric assay.

37. The device of claim 35, wherein detection of an amount of HD5 in the urine sample of at least 50 ng HD5/mg urine creatinine indicates the presence of a urinary tract infection in the subject.

38. The device of claim 35, wherein detection of an amount of HD5 in the urine sample of at least 25 ng HD5/mg urine creatinine indicates the presence of a urinary tract infection in the subject.

39. The device of claim 35, wherein the urinary tract infection is cystitis, pyelonephritis, or urethritis.

40. A kit comprising (i) components for the collection of a urine sample from a subject, (ii) components for detection of HD5 in the sample, wherein the components include a solid support coupled with an antibody to HD5, and (iii) instructions for use.

41. The kit of claim 40, wherein the kit indicates the presence or absence of a urinary tract infection in the subject based on the amount of HD5 detected in the sample.

42. The kit of claim 40, wherein the amount of HD5 in the sample is determined by at least one of an immunoassay, a colorimetric assay, a turbidimetric assay, or flow cytometry.

43. The kit of claim 40 wherein the components for identification of HD5 in the sample include gold beads or dyed beads wherein the beads are coupled to antibodies to HD5.

44. The kit of claim 41, wherein detection of an amount of HD5 in the urine sample of at least 50 ng HD5/mg urine creatinine indicates the presence of a urinary tract infection in the subject.

45. The kit of claim 41, wherein the urinary tract infection is cystitis, pyelonephritis, or urethritis.

46. A method for assessing if a subject is at risk of having or has a urinary tract infection, the method comprising the step of, determining if an HD5 level in a urine sample of the subject is above a threshold value using the device of claim 33, wherein an HD5 level above the threshold value indicates the subject is at risk of having or has a urinary tract infection.

47. The method of claim 46 wherein the threshold value is at least 50 ng HD5/mg urine creatinine.


SEQUENCE LISTING (2 sequences)

SEQ ID NO: 1 - DNA, 21 bp, Artificial Sequence
  Description of Artificial Sequence Synthetic DEFA5 forward primer
tccctcctgcaggtgacccca

SEQ ID NO: 2 - DNA, 23 bp, Artificial Sequence
  Description of Artificial Sequence Synthetic DEFA5 reverse primer
gtggctcttgcctgagaacctga